(19) United States
(12) Patent Application Publication
(10) Pub. No.: US 2006/0014861 A1
(43) Pub. Date: Jan. 19, 2006

(54) Compositions and methods relating to an adhesive composition

(75) Inventors: Gill G. Geesey, Bozeman, MT (US); Anthony P. Haag, Bozeman, MT (US)

Correspondence Address: Dorsey & Whitney LLP;Intellectual Property Department, Suite 1000, 555 California Street, San Francisco, CA, 94104-1513, US

(73) Assignee: Montana State University

(21) Appl. No.: 11/149,981

(22) Filed: Jun. 9, 2005

Related U.S. Application Data
(60) Provisional application No. 60/578,988, filed on Jun. 11, 2004.

(51) Int. Cl. A61K 9/16; C04B 37/00

(52) U.S. Cl. 524/56; 156/325

(57) ABSTRACT

The present invention is an adhesive composition including a biopolymer and methods of making and using the composition. The biopolymer in the composition can, in some instances, be a polysaccharide. The composition can also include a surfactant. In some embodiments, the polysaccharide is treated to reduce its hydrophilicity to improve moisture resistance of the adhesive bond.

### CROSS-REFERENCE TO RELATED APPLICATION

This application claims priority to U.S. Provisional Application No. 60/578,988, filed on Jun. 11, 2004, which is hereby incorporated herein by reference in its entirety.

### FIELD OF THE INVENTION

The present invention relates to an adhesive composition including a biopolymer and methods of making and using the composition. The present invention further relates to an adhesive composition including a polysaccharide.

### BACKGROUND OF THE INVENTION

Adhesives currently used in the wood products industry are petrochemically-based (phenol-formaldehyde, polyurethane, polyvinylacetate) adhesives and many also contain volatile organic compounds (VOCs) or other toxic chemicals. In the United States, greater than 1 billion pounds/year of these resins are utilized for wood products manufacturing. Minimization of VOC emissions and dependence on foreign petrochemical resources are driving the development of adhesives composed of alternative materials, including natural products-based adhesives.

Accordingly, there is a need in the art for an adhesive that contains no VOCs or other toxic chemicals and can be produced from a renewable resource.

### BRIEF SUMMARY OF THE INVENTION

The present invention, in one embodiment, is an adhesive composition. The composition has from about 10% by weight to about 50% by weight of dextran and from about 90% by weight to about 50% by weight of a solvent. The dextran, in one aspect of the invention, can be a dextran mixture including a dextran having an average molecular weight of about 500 kDa and a native dextran having a molecular weight ranging from about 2,000 to about 5,000 kDa. The composition can also have a surfactant. The present invention further includes a method of making the composition.

The present invention, in another embodiment, is a method of using an adhesive composition. The method includes applying the composition of claim 1 to a first substrate and contacting the first substrate with a second substrate. The method may, in one aspect of the invention, also include applying the adhesive composition to the second substrate before contacting the first substrate with the second substrate. In addition, the method may include subsequently applying moisture to the composition.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a graph depicting the effect of set time on shear strength of a sample at 23% (upper curve) and 53% (lower curve) RH. The error bars represent ±one standard deviation.

FIG. 2 is a graph depicting the effect of molecular weight on shear strength.

FIG. 3 is a graph depicting the effect on shear strength of the ratio of dextran500 DP and native polymer in a composition.

FIG. 4 is a graph depicting the effect on shear strength at high relative humidity of the ratio of dextran 500 DP and native polymer in a composition.

FIG. 5 is a graph depicting the effect of prolonged exposure to high (94%) RH on the strength of the bond formed with dextran (SB), O-acetylated dextran (SB-OAc), and Titebond™ on maple substrates after an initial one-week set period at moderate (53%) RH (5 replicates for each data point).

### DETAILED DESCRIPTION

The methods and compositions of the present invention are directed to an adhesive for use in various applications, including binding of wood or paper-based products. The compositions of the present invention relate to adhesive compositions comprising a biopolymer. Certain of the compositions are aqueous solutions having the biopolymer and a solvent, principally, water. The biopolymer can include, but is not limited to, a polysaccharide such as dextran or other microbial extracellular polysaccharides. The compositions can also include a surfactant, including surfactants produced by microorganisms. Additionally, the polysaccharide can be treated or derivatized to reduce its hydrophilicity, thereby increasing its resistance to moisture. Alternatively, certain compositions are removable by rehydration with an aqueous solution such as water, thereby facilitating recycling and/or decomposition of the composition or its components. Other embodiments of the compositions have mixtures of polysaccharides of different molecular weights. Further, the present invention also relates to methods of making and using the adhesive composition, including the use of certain embodiments of the composition to bind such wood products as, for example, maple, Douglas fir, particleboard, plywood, and fiberboard.

Unlike many known synthetic chemical adhesives used presently, biopolymer-based adhesive compositions contain no VOCs. Further, the biopolymer-based adhesive compositions can be biologically synthesized through fermentation of organic waste products, while presently-available commercial adhesives contain components derived from non-renewable petroleum-based products. Biopolymer-based adhesive compositions are also environmentally compatible and biodegradable and can be efficiently produced on a large-scale basis.

In one aspect, the present invention is a composition having a biopolymer. The term “biopolymer” is known in the art and is intended to include polysaccharides, proteins, and polyesters, and various combinations thereof. The biopolymer, in one embodiment, can be placed in aqueous solution. According to one embodiment, the concentration of the biopolymer in the composition is in an amount ranging from about 10% by weight to about 50% by weight. Alternatively, the concentration ranges from about 20% by weight to about 40% by weight. In a further alternative, the concentration is about 33%.

The composition in aqueous solution form includes an aqueous solvent. In one embodiment, the solvent is water. Alternatively, the solvent can be or can further contain any known solvent capable of use in an adhesive composition.

The biopolymer, in accordance with one aspect of the present invention, is a polysaccharide. The polysaccharide can be dextran. Alternatively, the polysaccharide is sodium carboxymethyl cellulose (CMC), dextrin, sodium alginate, or any known extracellular polysaccharides produced from any number of different microbes.

“Dextran” is a microbially-produced polysaccharide polymer which is composed of α-1,6-glucose. According to one embodiment, dextran is composed of repeating units of α-1,6-glucose. “Dextrin,” in comparison, is derived from starch (poly[alpha-1,4-glucose]) by thermal treatment which reduces its molecular weight and viscosity in water. As is known in the art, dextrans consist of linear chains of D-glucose subunits connected by alpha, 1-6 linkages. Although the number of glucose subunits in a polymer chain can vary, and thus display different sizes and molecular weights, all are considered to be the polysaccharide referred to as dextran. According to one embodiment, certain compositions of the present invention can contain any dextran within the average molecular weight range of from about 40 kDa to about 40,000 kDa. Alternatively, compositions of the present invention can contain any dextran within an average molecular weight range of from about 100 kDa to about 1,000 kDa. In a further alternative, compositions can contain a dextran found within an average molecular weight range of from about 400 kDa to about 600 kDa. A wide variety of dextrans are commercially available. For example, dextran from Leuconostoc mesenteroides can be obtained from Sigma Chemical Co. and has an average molecular weight of 500 kDa.

In a further embodiment, a composition of the present invention can comprise mixtures of various types of dextran and/or other polysaccharides, e.g., a composition can comprise more than one size fraction of dextran. For example, a composition according to one embodiment includes dextran having an average molecular weight of about 500 kDa (which, for purposes of this application, can also be referred to as “dextran500”) and native dextran (in this context, “native dextran” contains polymer chain lengths whose molecular weights range from about 2000-5000 kDa) The ratio of dextran500 to native dextran in compositions of the present invention can range from about 100:1 to about 1:100. Alternatively, the ration is about 100:1 to about 1:1. In a further alternative, the ratio is about 2:1 of dextran 500 to native dextran. In yet another alternative, the composition can contain any combination of dextrans of differing molecular weights. In accordance with one aspect of the invention, compositions having combinations of dextran500 and native dextran can have an effective binding strength that is equal to or greater than the binding strength of a composition having only one form of dextran.

Alternatively, a composition of the present invention can comprise any type of dextran in combination with any other polysaccharide. Examples of other polysaccharides include, but are not limited to, sodium carboxymethyl cellulose, dextrin, or sodium alginate. Sodium carboxymethyl cellulose is produced by alkylation of some of the hydroxyl groups of cellulose (poly[beta-1,4-glucose]) with chloroacetic acid. As discussed above, dextrin is derived from starch (poly[alpha-1,4-glucose]) by thermal treatment which reduces its molecular weight and viscosity in water. Sodium alginate is produced by plants and microorganisms and is a copolymer of guluronic and mannuronic acids. According to one embodiment, adhesive compositions of the present invention can contain any combination in any ratio of the above polysaccharides.

In accordance with one aspect of the present invention, the polysaccharide in the adhesive composition can be treated or derivatized with hydrophobic groups to reduce the hydrophilicity of the polysaccharide as compared to underivatized polysaccharides. The polysaccharide can be treated by any known method for reducing hydrophilicity. According to one embodiment, the polysaccharide is acetylated. For purposes of this application, “acetylation” is intended to include “partial acetylation” and “acetylated” is intended to include “partially acetylated.”

According to one embodiment, the acetylated derivatives of the polysaccharide have a degree of substitution (ratio of acetylated to non-acetylated) ranging from about 0.1 to about 3 hydroxyl groups. Alternatively, the degree of substitution ranges from about 1 to about 2. As is understood in the art, three is the maximum degree of substitution.

The acetylation process can occur by any known method. For example, the acetylation of dextran according to one embodiment employs acetic anhydride in varying ratios to control the degree of substitution. Alternatively, acetylation of the polysaccharides may occur biosynthetically by microbially-mediated derivitization.

A composition of the present invention including an acetylated derivative of a polysaccharide, in accordance with one embodiment, has increased moisture resistance in comparison to a composition having an untreated polysaccharide. Further, according to one aspect of the invention, the composition having an acetylated polysaccharide can provide shear strengths that are superior to non- treated compositions under conditions of high humidity, while exhibiting no loss of shear strength in conditions of moderate humidity.

The adhesive composition, according to an alternative aspect of the present invention, can also include a surfactant. According to one embodiment, the surfactant is present in the composition in an amount ranging from 0.1% by weight to about 5% by weight of the solids. Alternatively, the surfactant is present in an amount ranging from about 0.5% by weight to about 3% by weight of the solids. In a further alternative, the amount of surfactant in the composition is about 1% by weight of the solids.

A “surfactant” is a surface active agent that modifies the nature of surfaces, for example altering, e.g., reducing, the surface tension of water. There are generally four types of surfactants: cationic, anionic, nonionic and ampholytic, any of which can be used in the present invention, as well as mixtures thereof. In one aspect of the invention, the surfactant is a microbially-produced glycolipid. The glycolipid can be a monorhamnolipid or a dirhamnolipid. Alternatively, the surfactant is Tween, such as Tween-80. In a further alternative the surfactant is any known surfactant or mixtures thereof that can be included in an adhesive composition.

In an alternative embodiment of the present invention, the adhesive composition can also, as is understood in the art, include other elements, such as salts, buffers, stabilizers, antimicrobial preservatives, fillers, extenders, etc. That is, the composition can include any known element that may add to the effectiveness of the composition or may add additional benefits to the composition.

In one aspect of the present invention, the composition is made in the following manner. A culture of the bacterium Leuconostoc mesenteroides is grown in batch in a sucrose-containing liquid broth at room temperature until it has reached stationary phase. The bacterial cells are sedimented by centrifugation, and the cell-free culture fluid is treated with cold isopropyl alcohol to precipitate the dextran. The dextran is redissolved in deionized water, dialyzed against water to remove residual isopropyl alcohol, then dehydrated by lyophilization.

In use, the composition of the present invention is applied to a substrate to be bonded with another substrate. Alternatively, the composition is applied to both substrates. In a further aspect of the invention, more than two substrates are to bonded to each other and the composition is applied to one or more of the substrates. According to one embodiment, the substrates are wood. By “wood” herein is meant any number of woods and wood products, including hardwoods, softwoods, particle board, plywood, fiberboard, composites, wood laminates, etc. Depending on the use, the adhesive composition of the present invention is applied to any or all surfaces of one or more of the wood pieces to be bonded together. The surface may be rough or smooth. In some cases the surface may be prepared as needed, for example through the use of a planar, a sander, etc.

The composition, in accordance with one aspect of the invention, results in a relatively strong bond between the substrates. The composition results in a bond having an average bond strength of at least about 10 megapascals (“MPa”). One MPa is equal to 145.037738 pounds per square inch (“psi”). Alternatively, the average bond strength is at least about 11.7 MPa. In a further alternative, the average bond strength is at least about 13.7 to 18.3 MPa.

Alternatively, the composition results in a weaker bond between the substrates. According to one embodiment, the composition results in a bond having an average bond strength of less than about 3.447 MPa (about 500 psi).

In one aspect of the use of the invention, the binding properties of the adhesive composition can be reversed. That is, as explained above, certain forms of the composition having untreated polysaccharides are susceptible to moisture such that the application of moisture to the composition causes the binding strength of the composition to be reduced or eliminated. Thus, according to one embodiment, the composition can be applied to one or both substrates, the substrates placed in contact to create a bond between them, and then subsequently, moisture can be applied to the composition to weaken the bond to the point that the substrates can be separated.

Although the present invention has been described with reference to preferred embodiments, persons skilled in the art will recognize that changes may be made in form and detail without departing from the spirit and scope of the invention.

### EXAMPLES

The following examples are presented by way of demonstration, and not limitation, of the invention. Unless indicated otherwise, the following testing procedures were employed:

Shear strength is a measure of adhesive strength that is defined herein as a measure of the break strength of the adhesive composition along the plane of the bond. Shear strength is measured in accordance with ASTM D 905-94 (“Standard Test Method for Strength Properties of Adhesive Bonds in Shear by Compression Loading”). Generally, the method involves gluing two rectangular blocks of wood together with a 3 square inch bonded surface area and measuring the break strength in the direction along the plane of the bond. If the bond is sufficiently strong, its measurement may be limited by the strength of the wood substrate itself and it will show failure in the wood matrix.

### Example 1

Methods and Materials

The following experiments involved several different embodiments (“samples”) of the present invention, comparing (1) shear strength of 7 samples and one commercially-available wood glue (Experiment 1), (2) shear strength of one sample relative to a commercially-available wood adhesive using 4 different wood products (Experiment 2), (3) shear strength of one sample at different solids content (Experiment 3), (4) shear strength of one sample over time (Experiment 4), and (5) shear strength as a function of polysaccharide chain size (Experiment 5).

Sample 1 was an adhesive composition having dextran from the bacterium Leuconostoc mesenteroides. The dextran in this sample exhibited an average molecular weight of 500 kDa and was obtained from Sigma Chemical Co. (product no. D-5251). Herein, this sample is also referred to as “dextran500S” or “SB”.

Sample 2 was an adhesive composition containing partially-acetylated dextran500S (herein referred to as “Dextran-OAc or SB-OAc”). The sample was prepared in the following manner. Dried dextran500S (5.10 grams [g], containing 4.5% water, MW 505,000) was dissolved in 9.59 g of water. Pyridine (82 mL) was added and then, with efficient stirring, acetic anhydride (41.50 g, 13.5 eqivalents) was added over one hour [h]. During the addition, temperature was maintained at <30° C. with a cooling bath. After stirring at ambient temperature for an additional 21 h, the reaction mixture was precipitated into 1.4 liters [L] of cold water. This mixture was allowed to settle while cooling at 5° C. for 3 h and then centrifuged to aid phase separation. The white, sticky precipitate was separated by decantation of the supernatant liquid. The precipitate was washed twice with 50 milliters [mL] of water and finally allowed to stand overnight with 50 mL of water. After decantation of the water, the soft, white gummy material weighed 15.91 g. After air-drying, its final weight was 7.51 g. IR and NMR spectra were then obtained: IR 1758 cm⁻¹; ¹H-NMR δ2.02 ppm (—COCH+EE₃); ¹³C-NMR δ169.3-170.0 (—COCH₃) and 20.4-21.2 ppm (—COCH₃); T_{g}=175° C. The degree of substitution of acetyl groups, as determined by the method of Hestrin using glucose pentaacetate as standard, was 58% (wt/wt) of theoretical maximum, which corresponds to a dextran formula of [glucose(OAc)_{1.73}]ₙ. Sample 2 was then prepared at a 36% solids concentration (wt/wt) in 44% aqueous ethanol.

Sample 3 was an adhesive composition including dextran composed of polymers chains ranging in molecular weight from 5,000 kDa to 40,000 kDa (herein, referred to as “high molecular weight dextran” or “5-40M”) High molecular weight dextran was obtained from Pharmachem Corp, Bethlehem, Pa. A 20% (wt/wt) concentration of native dextran was used in order to achieve a viscosity that compared to Sample 1.

Sample 4 was an adhesive composition including sodium alginate. Sodium alginate is produced by plants and microorganisms and is a copolymer of guluronic and mannuronic acids. The sodium alginate used in this example was product number A-2158, supplied by Sigma Chemical Company. A 17% (wt/wt) concentration of alginate was used in order to achieve a viscosity that compared to Sample 1.

Sample 5 was an adhesive composition including sodium carboxymethyl cellulose. Sodium carboxymethyl cellulose (herein referred to as Na—CMC) is produced by alkylation of some of the hydroxyl groups of cellulose (poly[α-1,4-glucose]) with chloroacetic acid. The Na—CMC used in this example was product no. C-8758 from Sigma Chemical Company. A 20% (wt/wt) concentration of Na—CMC was used in order to achieve a viscosity that compared to Sample 1.

Sample 6 was an adhesive composition including dextrin. Dextrin is derived from starch (poly[α-1,4-glucose]) by thermal treatment which reduces its molecular weight and viscosity in water. The dextrin used in this example was product no. 0161-17, supplied by Difco Company. A 50% (wt/wt) concentration of dextrin was used in order to achieve a viscosity that compared to Sample 1. The dextrin was heated to 80° C. to form a homogeneous paste and applied to the wood substrates before cooling.

Sample 7 was an adhesive composition including pullulan, a polysaccharide produced by a fungus, and was obtained from Sigma Chemical Company (product no. P4516).

Sample 8 was an adhesive composition including dextran (herein, also referred to as “dextran500DP” or “500K”) that was composed of polymers of similar (505 kDa) molecular weight range as Sample 1, but was obtained from Dextran Products Limited, Scarborough, Ontario, Canada (product no. Dex500DP).

Titebond™. Original Wood Glue (Franklin International; Columbus, Ohio) was obtained from a commercial supplier.

The substrates utilized for the experiments in this example were: hard maple, Douglas fir, particleboard, and medium density fiberboard. These boards were purchased from commercial lumber suppliers and cut to 12×2.5×0.75 inch dimensions. In addition, the maple and fir boards were freshly surfaced with a planer on the face to which the adhesive was to be applied.

Preparation of the boards for testing included the following: four pairs of 0.34 inch diameter holes were drilled at intervals along the length of each of the 12 inch boards so that they could be cut into five 2 inch long pieces after curing. The boards for testing were equilibrated at 53% relative humidity for 14 days prior to testing.

The testing procedures for the boards was as follows. After application of adhesive, the boards were closed and bolted together overnight. Open time was approximately 5 min. After removal of the bolts, curing continued for a total of 7 days at 53% relative humidity (RH) at 22° C. Typically during the sixth day of curing, the boards were then cut to 2 inch widths and 2 inch lengths, and 0.25 inch rabbet grooves were cut along each end to the glue line as in ASTM D 905-94. The test specimens were stored in plastic bags during transport to cutting and testing locations to minimize changes in moisture content.

Shear strength was determined according to ASTM D 905-94 using an Instron (model 4206) with load cell A509-5 rated at 30,000 lb capacity at a load rate of 0.2 in/min. Typically 5 to 10 replicates were performed for each experiment.

Experiment 1—Comparison of Shear Strength of Adhesive Containing Dextran with Adhesives Containing Other Polysaccharides and with Titebond™ Original Wood Glue

In Experiment 1, the shear strength of bonds formed on maple substrates with Samples 1-7 and Titebond™ cured at 53% RH, were compared. The adhesives were prepared as aqueous mixtures at a concentration which achieved comparable viscosities and easily applied with a brush.

Results

The results are shown in Table 1.

TABLE 1
Comparison of Shear Strength of Adhesives Containing
Polysaccharides to Titebond ™

| | | | Average | | |
|---|---|---|---|---|---|
| | | | strength | | |
| Entry | Adhesive | % solids | (MPa) | replicates | CV % |
| 1 | Titebond ™ | 45 | 19.7 | 10 | 11 |
| 2 | Sample 1 | 33 | 18.8 | 10 | 18 |
| 3 | Sample 2 | 33 | 13.8 | 5 | 17 |
| 4 | Sample 3 | 20 | 5.8 | 5 | 75 |
| 5 | Sample 4 | 17 | 9.0 | 4 | 21 |
| 6 | Sample 5 | 20 | 16.9 | 5 | 22 |
| 7 | Sample 6 | 50 | 4.1 | 5 | 58 |
| 8 | Sample 7 | 33 | 13.6 | 5 | 28 |

Of all the polysaccharide formulations tested, dextran500-containing adhesive (Sample 1) exhibited a sheer strength that most closely approached Titebond™. The adhesives containing other polysaccharides, including dextran-OAc and high molecular weight dextran, displayed shear strengths that were less than the dextran500-containing adhesive and Titebond™.

Experiment 2—Shear Strength Comparison of Dextran-Containing Adhesive and Titebond™ with Different Wood Products

In Experiment 2, the shear strength of bonds formed on different wood products with Sample 1, prepared as a formulation with a total solids content of 33% (wt/wt) was compared to bonds formed with Titebond™.

Results

The results are shown in Table 2.

TABLE 2
Shear Strength (MPa) of Dextran500 and Titebond ™ with
Different Wood Products

| | Maple | Douglas fir | MDF ^{a} | PB ^{b} |
|---|---|---|---|---|
| Sample 1 | 14.6 | 12.5 | 2.2 | 3.7 |
| | (5, 15%) | (5, 10%) | (4, 30%) | (5, 9%) |
| | (80% | (mostly | (cohesive | (cohesive |
| | cohesive | wood | failure) | failure) |
| | failure) | failure) | | |
| Titebond ™ | 18.5 | 13.4 | 4.0 | 4.2 |
| | (5, 18%) | (5, 10%) | (5, 10%) | (5, 6%) |
| | (80% | (wood | (wood | (wood |
| | adhesive | failure) | failure) | failure) |
| | failure) | | | |

^{a} medium density fiberboard,
^{b} particle board
(Number of replicates and coefficient of variation (standard deviation/mean × 100) are shown in parenthesis)

Sample 1 was nearly as strong as Titebond™ on maple and Douglas fir substrate when set at 53% relative humidity (RH) for 14 and 8 days, respectively at 22° C. (Table 2). The mode of failure for maple specimens was primarily cohesive failure in the case of Sample 1, whereas, it was primarily adhesive failure for Titebond™. The mode of failure for fir specimens was wood failure for both types of adhesive. In the case of MDF and PB specimens bonded with Sample 1, cohesive failure was the primary mode, whereas, those bonded with Titebond™ wood failure was the primary mode.

Experiment 3—Determination of Shear Strength of Adhesive Containing Different Dextran Solids Content

In Experiment 3, Sample 8 was prepared at different solids content, applied to maple substrates, and after curing for 8 days at 53% RH, the shear strength of the specimens evaluated.

Results

The results are presented in Table 3.

TABLE 3
Effect of Polysaccharide Concentration in Adhesive Composition
on the Shear Strength of Bonded Maple Surfaces.

| | Average shear | | |
|---|---|---|---|
| % solids (wt/wt) | strength (MPa) | Replicates | CV % |
| 50 | 14.6 | 4 | 28 |
| 40 | 13.7 | 5 | 15 |
| 33 | 15.0 | 5 | 12 |

The different solids content between 33-50% yielded similar adhesive strengths. The variability between replicates increased as concentration increased. The results suggest that a 33% total solids content offers high shear strength with good replicability.

Experiment 4—Examination of Shear Strength of Dextran500-containing Adhesive over Time

In Experiment 4, the bond strength of Sample 1, prepared as a 33% (wt/wt) formulation, was examined over time at 23% and 53 % RH using maple substrates.

Results

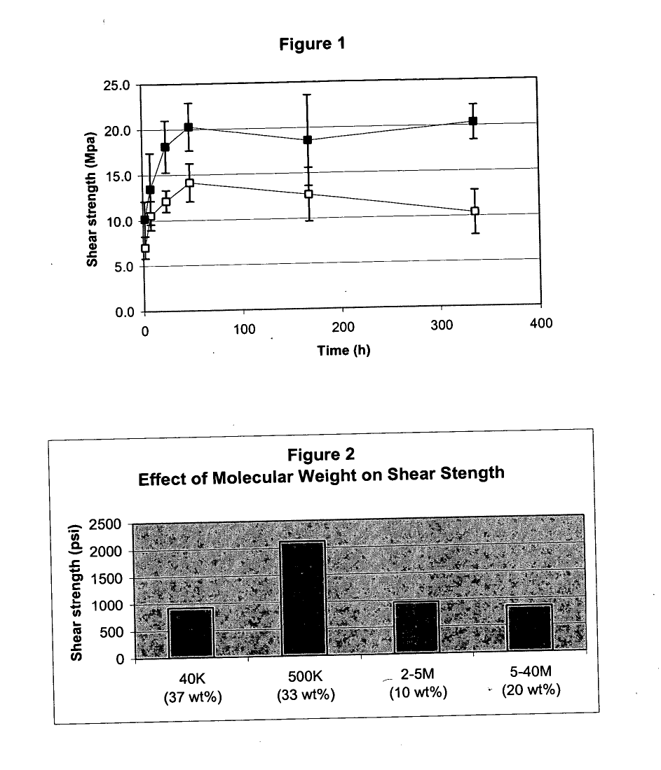

The results are shown in FIG. 1.

At 53% RH, half (7.0 MPa) of the maximum shear strength was obtained in two hours (FIG. 1). The maximum shear strength (14.1 MPa) was obtained in two days and then shear strengths decrease slowly over time. Maximum shear strength was 50% higher when cured at 23% RH than at 53% RH (FIG. 1). At 23% RH, half (10.2 MPa) of the maximum shear strength was also attained within two hours of joining and the maximum (20.2 MPa) was obtained in 48 h.

### Example 2

Methods and Materials

The following experiments involved several different embodiments (“samples”) of the present invention, comparing (1) shear strength of 4 samples of adhesive containing dextran containing polymers of different molecular weight and (2) shear strength of three samples containing different amounts of native dextran with a molecular weight range of 2,000-5,000 kDa and dextran with an average molecular weight of 505 kDa.

Sample 1 (herein, also referred to as “dextran500DP” or “500K”) was an adhesive composition identical to Sample 8 in Example 1 above which contained dextran that was composed of polymers of average molecular weight of 505 kDa.

Sample 2 (herein, also referred to as “40K”) contains a dextran whose polymers have a molecular weight of 41 kDa. This dextran was obtained from Sigma Chemical Co. (product no. D-1662).

Sample 3 (herein, referred to as “2-5M” or “native polymer”) contains a dextran whose polymers have a molecular weight range of 2,000-5,000 kDa. This dextran (product no. native, grade 2P) was obtained from Pharmachem Corp.

Sample 4 (herein, also referred to as “high molecular weight dextran”) is the same as Sample 3 in Example 1 above. This dextran sample is composed of polymers chains ranging in molecular weight from 5,000-40,000 kDa (herein, referred to as “high molecular weight dextran or “5-40M”) High molecular weight dextran was obtained from Pharmachem Corp, Bethlehem, Pa.

Experiment 1—Effect of Polysaccharide Molecular Weight on Shear Strength of Dextran-Containing Adhesive

In Experiment 1, shear strength of bonds formed with maple substrates using four adhesives containing dextran polymers of different molecular weight were compared after applying to maple substrates and curing at 53% RH at 22° C. for 1 week. Since Samples 3 and 4, contained higher molecular weight polymers than Samples 1 and 2, they formed more viscous solutions at comparable concentrations than the latter. For example, whereas it was easy to form solutions of Samples 1 and 2 that could be applied to substrates at total solids content of 33-40%, (wt/wt), such was not possible with Samples 3 and 4. Therefore, adhesive Samples 3 and 4 were prepared at lower concentrations (10-20%, wt/wt) having viscosities similar to Samples 1 and 2.

Results

The results are set forth in FIG. 2.

The results show that adhesive containing dextran polymers with a molecular weight of 500 kDa forms bonds with greater shear strength than adhesive containing dextran polymers with higher or lower molecular weight.

Experiment 2

In Experiment 2, the shear strength of bonds formed on maple specimens using three adhesive compositions containing varying amounts of native polymer (2-5M) and dextran500DP (500K) were compared after curing at 53% RH at 22° C. for 2 weeks. Sample 1 was an adhesive containing dextran599DP, with a molecular weight of 500 kDa, prepared at a 33% (wt/wt) solids content. Sample 2 was a mixture of dextran500DP and native polymer (2-5M) at a total solids content of 33% with native polymer and dextran500DP contributing 20% and 80% of the total, respectively. Sample 3 was a mixture of dextran500DP and native polymer at a total solids content of 33% with native polymer and dextran500DP contributing 35% and 65% of the total, respectively.

Results

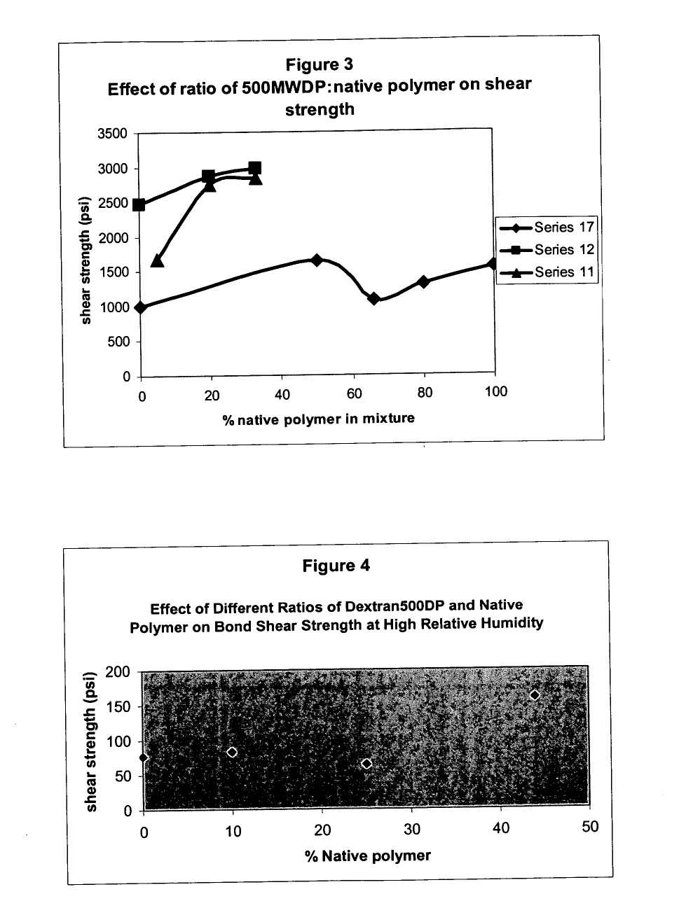

The results are presented as Series 12 in FIG. 3. The introduction of increasing amounts of native polymer with dextran500DP into the blend produced an adhesive with higher shear strength than that achieved by the adhesive containing only the dextran500DP.

Experiment 3

Experiment 2 was repeated except that the total solids content was 30% (wt/wt) for all three samples, native polymer and dextran500DP contributed 5 and 95% of the total solids, respectively, in Sample 1, and the samples were evaluated after curing for 1 week at 53% RH at 22° C.

Results

The data presented as Series 11 in FIG. 3 followed the trend observed in Series 12 data. As the amounts of native polymer increase from 5 to 33% of the total dextran present, shear strength of the adhesive increased.

Experiment 4

Experiment 2 was repeated again using an adhesive containing dextran at a total solids content of 20% (wt/wt) in order to allow evaluation of the effect of higher native polymer content relative to dextran500DP. Five samples were prepared: Sample 1 contained no native polymer (100% dextran500DP). Sample 2, 3, 4, and 5 contained native polymer that contributed 50%, 67%, 80% and 100% of the total solids, respectively, With the balance contributed by dextran500DP.

Results

The data are presented as Series 17 in FIG. 3. As in previous experiments, shear strength of the adhesive increased as the native polymer content increased between 0 and 50% of total solids. However, due to the lower total solids content of these samples, shear strength was significantly lower than that achieved with blends containing 30-33% total solids. Although the shear strength decreased as native polymer solid content increased from 50 to 66%, the difference in shear strength of adhesive mixtures containing no native polymer and those containing 100% native polymer was significant at p=0.02 level. The data from Experiments 2-4 suggest that highest shear strength can be achieved with an adhesive with a total solids content of 33% (wt/wt) consisting of 1 part native polymer to 2 parts dextran500DP.

### Example 3

Methods and Materials

The following experiments involved several different embodiments (“samples”) of the present invention, comparing (1) shear strength of one sample containing a surfactant in varying amounts and one sample that does not include a surfactant (Experiment 1), (2) shear strength of bonds formed on maple substrates by samples containing mixtures of dextran500DP and native polymer with and without surfactant, (3) shear strength of bonds formed on particleboard (PB) and medium density fiberboard (MDF) substrates by samples containing dextran500DP with and without surfactant.

Sample 1 (herein, also referred to as “dextran500DP” or “500K”) was an adhesive composition identical to Sample 8 in Example 1 above, and to Sample 1 in Example 2 above, which contained dextran that was composed of polymers of average molecular weight of 505 kDa.

Sample 2(herein, also referred to as “2:1 dextran500DP native polymer”) is an adhesive containing a total solids content of 33% (wt/wt) of which dextran500DP and native polymer contribute 67% and 33% of the total solids, respectively.

Two types of naturally occurring surfactants, a monorhamnolipid (mRL), obtained from R. Maier from the University of Arizona, Tucson, Ariz., and a mono/di-RL mixture (herein also referred to as “JBR RL”) (product no. JBR425) obtained from Jeneil Biosurfactant Company (Saukville, Wis.). (as shown in FIG. 2), were added to Sample 1. In addition, the surfactant Tween™ 80 (polyoxyethylene sorbitan monooleate) obtained from Fischer Scientific (Pittsburgh, Pa.) was also added to Sample 1.

Three types of substrates were tested: maple, particleboard (PB), and medium density fiberboard (MDF).

Experiment 1—Impact of Addition of Surfactants on Shear Strength of Dextran500-Containing Adhesive

In Experiment 1, the impact of various amounts and types of surfactant additives on the shear strength of Sample 1, prepared as a 40% (wt/wt) formulation was examined using maple substrates. Two types of naturally occurring surfactants, a monorhamnolipid (mRL) and a mono-dirhamnolipid mixture (JBR RL), were tested. The biologically-derived surfactants mRL and JBR RL were added to dextran500 at two concentrations: 0.1% and 1.0% (wt/wt adhesive solids). The surfactant Tween™ 80 was added to dextran500 at 0.1, 0.5, and 1.0% (wt/wt adhesive solids). The general structure of a rhamnolipid (Monorhamnolipid, R═H; dirhamnolipid, R═L-rhamnosyl) is as follows:

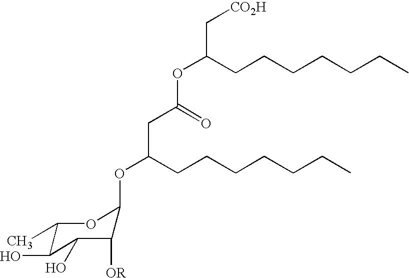

Results

The results are shown in Table 4.

TABLE 4
Effect of Surfactants on Shear Strength
of Dextran500-Containing Adhesive

| | Surfactant | Average | | |
|---|---|---|---|---|
| | concentration | strength | | |
| Adhesive composition | (wt % ^{a}) | (MPa) | Replicates | CV % |
| Sample 1 | 0 | 13.7 | 5 | 49 |
| Sample 1 + mRL | 0.1 | 16.5 | 5 | 9 |
| Sample 1 + mRL | 1.0 | 17.9 | 4 | 15 |
| Sample 1 + JBR-RL | 0.1 | 14.5 | 5 | 19 |
| Sample 1 + JBR-RL | 1.0 | 11.3 | 5 | 24 |
| Sample 1 + Tween 80 | 0.1 | 15.0 | 5 | 9 |
| Sample 1 + Tween 80 | 0.5 | 14.3 | 5 | 14 |
| Sample 1 + Tween 80 | 1.0 | 14.6 | 5 | 14 |

^{a} solids basis

Addition of all surfactants at all concentrations tested, except for the mono-/dirhamnolipid mixture (JBR-RL) at 1.0% (wt/wt) solids content, enhanced shear strength of dextran500DP-containing adhesive. The greatest enhancement (23%) was achieved with the higher concentration of the monorhamnolipid (mRL). The shear strength of bonds formed with adhesives containing mRL at either concentration was significantly greater (p<0.05) than that achieved by adhesive without mRL). No significant difference was obtained for shear strength of bonds formed with adhesives containing the other surfactants and those without surfactant. A large amount of wood failure was observed at the bondline for specimens bonded with the adhesive containing dextran500DP and mRL. Where the bond failed (as opposed to the wood), all specimens showed cohesive failure. That is, the surface appeared to be well covered with adhesive on both faces of the wood adherend.

### Example 4

Materials and Methods

The following experiments involved several different embodiments (“samples”) of the present invention: (1) determining the influence of the ratio of dextran500DP and native dextran on shear strength of bonds exposed to high relative humidity after curing at moderate relative humidity (2) determining the influence of O-acetylation of dextran500DP-containing adhesives on shear strength of bonds exposed to high relative humidity over extended periods of time after curing at moderate relative humidity (3) comparison of underivatized and O-acetylated dextran500S and MB adhesive containing another microbial polysaccharide.

Moisture resistance was evaluated by applying different formulations of adhesive to maple substrates prepared as described in Example 1 above, clamping the adhesive-containing surfaces together and curing the specimens at 22° C., at 53% RH (herein, also referred to as “moderate RH”) for one week, followed by incubation for additional periods of time at 94% RH (herein, also referred to as “high RH”) at 22° C., and then testing the shear strength of the bonded specimens as described in Example 1 above. To achieve the desired relative humidity during curing, test specimens were incubated in a closed chamber containing saturated aqueous solutions of magnesium nitrate (53% RH) or potassium nitrate (94% RH).

Sample 1 was an adhesive composition having dextran500DP. The sample comprised a 33% (wt/wt) aqueous solution of dextran500DP.

Sample 2 was an adhesive composition having a total solids content of 33% (wt/wt) with dextran500DP and native polymer contributing 90% and 10%, respectively, of the total solids.

Sample 3 was an adhesive composition having a total solids content of 33% (wt/wt) with dextran500DP and native polymer contributing 75% and 25%, respectively, of the total solids.

Sample 4 was an adhesive composition having a total solids content of 33% (wt/wt) with dextran500DP and native polymer contributing 55% and 45%, respectively, of the total solids.

Sample 5 was an adhesive composition that contained partially acetylated dextran500S having a solids content of 36% (wt/wt)(Sample 2 in Example 1 above).

Sample 6 was an adhesive composition containing a microbial polysaccharide obtained from and proprietary to Montana Biotech, Inc., Belgrade, Mont. at a solids content of 33% (wt/wt) (“MB” or “MB adhesive”).

Sample 7 was an adhesive composition containing an O-acetylated derivative of sample 6 above (57% degree of O-acetylation) at a solids content of 33% (wt/wt).

Titebond™ Original Wood Glue was also used in some experiments as a metric.

The following experiment involved four different embodiments (“samples”) of the present invention, testing the shear strength of each.

Experiment 1—Influence of Ratio of Dextran500DP and Native Dextran on Shear Strength of Bonds Exposed to High Relative Humidity

In Experiment 1, adhesive compositions comprising varying amounts of native polymer (having a molecular weight of 2000-5000 kDa) and dextran500DP (having a molecular weight of 505 kDa) at a total solids content of 33% (wt/wt) were applied to maple substrates, the glued faces clamped together, and the specimens cured at 53% RH for one week followed by exposure to 94% RH for two weeks before evaluating shear strength of the bond.

Sample 1 was an adhesive formulation containing dextran500DP at a total solid content of 33% (wt/wt). Sample 2 was an adhesive formulation containing a mixture of 90% dextran500DP and 10% native polymer at a total solid content of 33% (wt/wt). Sample 3 was an adhesive formulation containing a mixture of 75% dextran500DP and 25% native polymer at a total solid content of 33% (wt/wt). Sample 4 was an adhesive formulation containing a mixture of 55% dextran500DP and 45% native polymer at a total solid content of 33% (wt/wt).

Results

The results are set forth in FIG. 4.

The shear strength of bonds formed on maple substrates with adhesive containing different ratios of dextran500DP and native polymer after 2 weeks exposure to 94% RH following a 1-week cure at 53% RH was very low compared to the shear strength exhibited by bonds formed by adhesive containing these different blends that were not exposed to the high humidity after curing (compare shear strengths in FIG. 4 with those presented in FIG. 3 of Example 2 above)

Experiment 2—Impact of Moisture on Shear Strength of Dextran and Dextran-OAc

In Experiment 2, the influence of high relative humidity, on shear strength of bonds formed on maples substrates with adhesive containing Sample 1 (dextran500DP or SB), Sample 2 (O-acetylated dextran500S or SB-OAc), and Titebond™ was determined. The specimens were cured at 53% (moderate) RH for one week, then half of the samples were held at 53% and half at 94% (high) RH for additional periods of time before testing bond strength.

Results

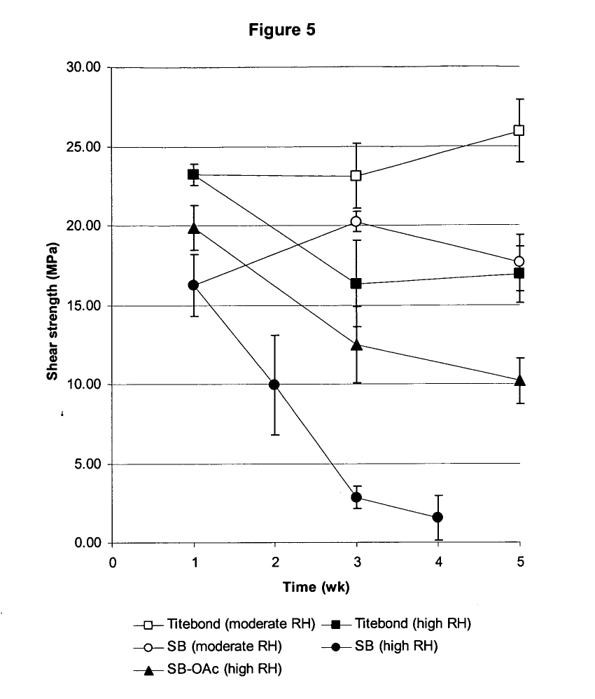

The results are shown in FIG. 5. When exposed to 94% RH after 1 week set period at 53% RH, Titebond™ retained 69% of the strength observed after the initial 1-week set at 53% RH, but before exposure to the higher RH (FIG. 5). After exposure to 94% RH for 1, 3 and 5 weeks, SB- or dextran500DP- containing adhesive retained 61%, 17% and 9%, respectively, of that observed after the initial 1-week set at 53% RH, but before exposure to the higher RH (FIG. 5). The results are consistent with a reversible setting mechanism in which shear strength is dependent on the concentration of water in the adhesive. Based on the evidence that bond strength is compromised by water absorption at high humidity, the dextran-containing adhesive was chemically modified to impart hydrophobic character. The polysaccharide was partially acetylated in which 58% of the available hydroxyl groups were substituted. When partially-acetylated dextran500S-containing adhesive (SB-OAc) was prepared as a 36% solution in aqueous ethanol and used to bond maple substrates, a significant improvement in moisture resistance was observed (FIG. 5). Bonds formed after one week at 53% RH exhibited a shear strength that was 122% of that formed by underivatized dextran500DP-containing adhesive (SB) and 86% of that formed by Titebond™ set under the same conditions (FIG. 5). After two weeks exposure at 94% RH, O-acetylated dextran500S-containing adhesive (SB-OAc) maintained a shear strength of 63% of its initial value. By comparison, bonds formed with underivatized dextran500DP-containing adhesive (SB) and Titebond™ maintained 15% and 69%, respectively, of the strength displayed under the same conditions (FIG. 5). After four weeks exposure at 94% RH, bonds formed with O-acetylated dextran500S-containing adhesive (SB-OAc) maintained 51% (10.2 MPa) of the shear strength displayed by bonds formed after 1 week at 53% RH (FIG. 5). By comparison, underivatized dextran500DP-containing adhesive (SB) and Titebond™ formed bonds that maintained 9% (1.5 MPa) and 70% (16.9 MPa), respectively, of the shear strength achieved after 1 week at 53% RH. Thus, substitution of dextran500S hydroxyl groups with more hydrophobic acetate esters effectively improves shear strength of bonds formed by adhesive containing this polymer at moderate and high RH. These results support earlier evidence that the strength of the bond formed between wood substrates and dextran500DP or dextran500S adhesive depends on water exclusion.

Experiment 3. Comparison of Underivatized and O-acetylated Dextran500S and MB Adhesive After Exposure to High Relative Humidity

In Experiment 3, shear strength of bonds formed with underivatized and O-acetylated dextran500S and MB adhesive on maple substrates were determined after setting for 1 week at 53% RH and either continued exposure to this relative humidity or exposure to 94% relative humidity.

Results

When shear strength of bonds formed with dextran 500S and MB adhesive on maple substrates were compared after setting for two weeks at 53% RH no significant difference was observed (Table 5). However the shear strength of dextran500S-bonded specimens after setting for 1 week at 53% RH followed by exposure to 94% RH for an additional week were significantly higher than the MB-bonded specimens (1537 psi vs 157 psi) (Table 5). Shear strengths of the bonds formed by both the dextran500S and MB adhesive with maple were similar after exposure to 94% RH for two weeks (6 psi vs. 0 psi) (Table 5).

The corresponding partially acetylated derivatives of dextran500S and MB adhesive were also compared. Dextran500S-OAc and MBOAc had degrees of acetylation of 58 and 57%, respectively. After a one-week set period at 53% RH, half of the specimens were continued for another two weeks at 53% RH and half were exposed to 94% RH for two weeks. In the comparison at three weeks continuous exposure at 53% RH, dextran500SOAc showed a significantly higher shear strength than MBOAc (3296 and 2512 psi, respectively) (Table 5). Under conditions of exposure for two weeks at 94% RH, dextran500SOAc displayed significantly higher shear strength (1835 psi) than MBOAc (799 psi) (Table 5). In summary, dextran500S maintained slightly higher shear strength than MB adhesive at 53% RH. Although both eventually lost all their strength at 94% RH, the rate of loss, or the sensitivity to moisture, was lower for dextran500S. The acetylated derivatives both showed improved moisture resistance in comparison to the underivatized materials, but dextran500SOAc maintained significantly higher shear strengths at both 53 and 94% RH than MBOAc.

TABLE 5
Effect of humidity on shear strength of bonds formed with underivatized
and O-acetylated dextran500S and MB adhesive on maple substrates

| | Weeks at | | | | | |
|---|---|---|---|---|---|---|
| | 94% RH | | % Change | | | |
| | after 1 | Ave. | at 94% RH | | | |
| | week set | strength | relative to | | | |
| Adhesive | at 53% RH | (psi) | 53% RH | Replicates | CV % | p-value* |
| Dextran500S | 0 | 2119 | | 5 | 15 | control |
| Dextran500S | 1 | 1537 | −27 | 5 | 28 | |
| MB | 0 | 2043 | | 4 | 13 | 0.36 |
| MB | 1 | 157 | −92 | 5 | 67 | |
| Dextran500S | 0 | 2968 | | 5 | 17 | control |
| Dextran500S | 2 | 96 | −97 | 5 | 44 | |
| MB | 0 | 2159 | | 4 | 20 | 0.017 |
| MB | 2 | 0 | −100 | 5 | | |
| Dextran500OAc | 0 | 3296 | | 5 | 6 | 0.12 |
| Dextran500OAc | 2 | 1427 | −57 | 5 | 21 | |
| MBOAc | 0 | 2512 | | 5 | 9 | 0.0002** |
| MBOAc | 2 | 799 | −68 | 4 | 12 | |

*Probability that shear strengths are not significantly higher than the control at the 95% confidence level determined by the student t-test (one-tail analysis).
**compared to dextran500SOAc

1. An adhesive composition comprising:
(a) from about 10% by weight to about 50% by weight of dextran; and
(b) from about 90% by weight to about 50% by weight of a solvent.

2. The composition of claim 1, wherein the dextran has an average molecular weight of from about 40 kDa to about 40,000 kDa.

3. The composition of claim 1, wherein the dextran comprises a dextran mixture comprising:
(a) a dextran having an average molecular weight of about 500 kDa; and
(b) a native dextran having a molecular weight ranging from about 2,000 to about 5,000 kDa.

4. The composition of claim 3, wherein the dextran mixture comprises a ratio ranging from about 100:1 to about 1:1 of the dextran having an average molecular weight of about 500 kDa to the native dextran.

5. The composition of claim 1, wherein the dextran is a derivatized dextran.

6. The composition of claim 5, wherein the derivatized dextran is an acetylated dextran.

7. The composition of claim 1, wherein the solvent comprises water.

8. The composition of claim 1, further comprising a surfactant.

9. The composition of claim 8, wherein the surfactant is present in the composition in an amount of from about 0.1% by weight to about 5% by weight with respect to the dextran.

10. The composition of claim 8, wherein the surfactant is chosen from the group consisting of a glycolipid, a monorhamnolipid, a dirhamnolipid, and Tween.

11. The composition of claim 1, wherein the composition has an average bond strength of at least 500 psi.

12. The composition of claim 1, wherein the dextran is from Leuconostoc mesenteroides.

13. The composition of claim 1, further comprising another polysaccharide.

14. A method of making the composition of claim 1, the method comprising:
growing Leuconostoc mesenteroides in a fluid comprising sucrose;
removing cells of Leuconostoc mesenteroides from the fluid; and
treating the fluid with isopropyl alcohol.

15. A method of using an adhesive composition comprising:
applying the composition of claim 1 to a first substrate; and
contacting the first substrate with a second substrate.

16. The method of claim 15 further comprising applying the adhesive composition to the second substrate before contacting the first substrate with the second substrate.

17. The method of claim 15, wherein the dextran comprises a dextran mixture comprising:
(a) a dextran having an average molecular weight of about 500 kDa; and
(b) a native dextran having a molecular weight ranging from about 2,000 kDa to about 5,000 kDa.

18. The method of claim 15, wherein the dextran is a derivatized dextran, wherein the derivatized dextran has reduced hydrophilicity.

19. A method according to claim 15-18 wherein at least one of the first substrate and the second substrate is chosen from the group consisting of a hard wood, a soft wood, and wood particle board.

20. The method of claim 15, further comprising subsequently applying moisture to the composition.

21. The method of claim 20, wherein the applying moisture causes a bond between the first substrate and second substrate to weaken.
